(19) United States
(12) Patent Application Publication
(10) Pub. No.: US 2016/0317070 A1
(43) Pub. Date: Nov. 3, 2016

(54) NON-INVASIVE BLOOD GLUCOSE MONITORING WITH A WEARABLE DEVICE

(71) Applicant: Ram Sivaraman, Dublin, CA (US)

(72) Inventors: RAM SIVARAMAN, DUBLIN, CA (US); Nandita Shankar, Cranford, NJ (US)

(73) Assignee: Ram Sivaraman, Dublin, CA (US)

(21) Appl. No.: 14/697,623

(22) Filed: Apr. 28, 2015

(51) Int. Cl. A61B 5/145; A61B 5/00; A61B 5/1455

(52) U.S. Cl. CPC A61B 5/14532; A61B 5/1455; A61B 5/6801; A61B 5/7282; A61B 5/747; A61B 5/0022

(57) ABSTRACT

A method for non-invasive blood glucose monitoring with a wearable device may include illuminating, with an electromagnetic source module of a wearable device, an area of skin of a user. The method may include measuring a scattered electromagnetic energy reflected from the area of skin with an electromagnetic receiver module of the wearable device. The method may include calculating, with a processor coupled to the electromagnetic receiver module, a blood glucose level in response to the measurement of the scattered electromagnetic energy reflected from the area of skin. The method may include communicating the blood glucose level to an information handling system. The electromagnetic source module may include a photodiode or a microwave module. The information handling system may include an application configured for a smart phone, tablet, or computer. The information handling system may include a memory configured for storing a historical record including multiple blood glucose levels.

### FIELD

This disclosure relates generally to blood analysis, and more specifically, to non-invasive blood glucose monitoring with a wearable device.

### BACKGROUND

People who suffer from diabetes need to accurately monitor their blood sugar (i.e., glucose) levels in order to help regulate their medications, diet, and exercise. Conventional blood glucose tests include an invasive blood sample collection method in which a lancet is used to draw blood for analysis in conjunction with a test strip. The lancet typically pricks a finger of the user and is thus considered to be invasive. Since many diabetics must test their blood glucose levels multiple times per day, these invasive “finger pricks” can be a major deterrent due to the cumulative effects of experiencing multiple finger pricks over time. Accurate and timely blood glucose measurements are crucial to the effective treatment of diabetes. Therefore any source of potential deterrent to regular testing, such as a child's aversion to sore fingers or an elderly adult's failing memory, may be detrimental to a patient's health.

As the value and use of information, such as medical records, continues to increase, individuals and businesses seek additional ways to process and store information. One option available to users is information handling systems. An information handling system generally processes, compiles, stores, and/or communicates information or data for business, personal, or other purposes thereby allowing users to take advantage of the value of the information. Because technology and information handling needs and requirements vary between different users or applications, information handling systems may also vary regarding what information is handled, how the information is handled, how much information is processed, stored, or communicated, and how quickly and efficiently the information may be processed, stored, or communicated. The variations in information handling systems allow for information handling systems to be general or configured for a specific user or specific use such as financial transaction processing, airline reservations, enterprise data storage, or global communications. In addition, information handling systems may include a variety of hardware and software components that may be configured to process, store, and communicate information and may include one or more computer systems, data storage systems, and networking systems.

### SUMMARY

Methods and systems for non-invasive blood glucose monitoring with a wearable device are described. In one embodiment a method may include illuminating, with an electromagnetic source module of a wearable device, an area of skin of a user. The method may include measuring scattered electromagnetic energy reflected from the area of skin with an electromagnetic receiver module of the wearable device. Additionally, the method may include calculating, with a processor coupled to the electromagnetic receiver module, a blood glucose level in response to the measurement of the scattered electromagnetic energy reflected from the area of skin. The method may further include communicating the blood glucose level to an information handling system.

In one embodiment, the information handling system may include a memory configured for storing a historical record including multiple blood glucose levels. The information handling system may further include a second processor configured to selectively transmit an alert to a physician in response to one of the multiple blood glucose levels exceeding an upper threshold level, one of the multiple blood glucose levels being below a lower blood glucose threshold level, or a slope of a plotted line of historical blood glucose levels being outside a pre-defined safe blood glucose rate of change. Additionally, the processor may be configured to automatically initiate a blood glucose test at pre-programmed time intervals, increase the frequency of the pre-programmed time intervals in response to one of the multiple blood glucose levels exceeding the upper blood glucose threshold level or being below the lower blood glucose threshold level, and increase the frequency of the pre-programmed time intervals in response to the slope of the plotted line of historical blood glucose levels being outside the pre-defined safe blood glucose rate of change. In an embodiment, the processor may initiate a blood glucose test in response to a user pressing a button coupled to the processor, or a user selecting a manual test initiation feature of an application of the information handling system. In one embodiment, the processor may be configured to calibrate the electromagnetic receiver module based on a pre-defined reference value. In various embodiments, the electromagnetic source module may include a photodiode module or a microwave module.

In one embodiment, a system for non-invasive blood glucose monitoring with a wearable device may include an information handling system. Additionally, the system may include a wearable device. In an embodiment, the wearable device may include an electromagnetic source module configured to illuminate an area of skin of a user. In one embodiment, the wearable device may include an electromagnetic receiver module configured to measure a scattered electromagnetic energy reflected from the area of skin. The wearable device may further include a processor configured to calculate a blood glucose level in response to the measurement of the scattered electromagnetic energy reflected from the area of skin. Additionally, the wearable device may include a communication module configured to communicate the blood glucose level to the information handling system. In one embodiment, the information handling system may include an application configured for a smart phone, tablet, or computer.

In an embodiment, an apparatus for non-invasive blood glucose monitoring with a wearable device may include an information handling system. Additionally, the apparatus may include a wearable device. In an embodiment, the wearable device may include an electromagnetic source module configured to illuminate an area of skin of a user. In one embodiment, the wearable device may include an electromagnetic receiver module configured to measure a scattered electromagnetic energy reflected from the area of skin. The wearable device may further include a processor configured to calculate a blood glucose level in response to the measurement of the scattered electromagnetic energy reflected from the area of skin. Additionally, the wearable device may include a communication module configured to communicate the blood glucose level to the information handling system.

### BRIEF DESCRIPTION OF THE DRAWINGS

The present invention(s) is/are illustrated by way of example and is/are not limited by the accompanying figures, in which like references indicate similar elements. Elements in the figures are illustrated for simplicity and clarity, and have not necessarily been drawn to scale.

FIG. 1 is a schematic block diagram illustrating one embodiment of a system for non-invasive blood glucose monitoring with a wearable device.

FIG. 2 is a schematic block diagram illustrating one embodiment of an Information Handling System (IHS) configured for non-invasive blood glucose monitoring with a wearable device.

FIG. 3 is a schematic flowchart diagram illustrating one embodiment of a method for non-invasive blood glucose monitoring with a wearable device.

FIG. 4 is a schematic block diagram illustrating one embodiment of an apparatus for non-invasive blood glucose monitoring with a wearable device.

FIG. 5 is a schematic block diagram illustrating one embodiment of an apparatus for non-invasive blood glucose monitoring with a wearable device.

### DETAILED DESCRIPTION

Embodiments of methods and systems for non-invasive blood glucose monitoring with a wearable device are described. In an embodiment, a wearable device for non-invasive blood glucose monitoring may include an electromagnetic energy source configured to illuminate an area of the user's skin, an electromagnetic detector that receives the scattered electromagnetic energy reflected by the area of skin, a processor that calculates a blood glucose level based on the reflected energy, and an information handling system configured to wirelessly receive the calculated blood glucose level data.

As utilized herein, the term “electromagnetic energy” means a measureable energy having a wavelength in the electromagnetic spectrum, including, but not limited to, visible light, infrared energy, ultraviolet energy, microwave energy, high frequency energy, radio waves, or photons.

In an embodiment, a wearable device is positioned in proximity to a user's skin in an area where the tissue includes more vascular or muscle content than fat (e.g., on a wrist or an arm) to increase the accuracy of the measurements. The wearable device may include a processor, a battery power source, a memory module, a wireless communication module, an electromagnetic source module, and an electromagnetic receiver module. In one embodiment the processor may automatically initiate a blood glucose test at periodic intervals by signaling the electromagnetic source module to illuminate and/or radiate an area of the user's skin. In various embodiments the electromagnetic source may shine visible light, emit infrared energy, or radiate microwave energy towards the area of skin such that the energy penetrates the skin and reaches the blood and interstitial fluid in the capillary region of the skin. The relative level of glucose in blood and interstitial fluid alters the electromagnetic signature of the blood and interstitial fluid. For example, glucose raises the refractive index of interstitial fluid, thereby decreasing the scattering coefficient of the tissue. Similarly, glucose lowers the dielectric constant of blood, thereby affecting the capacitance and resonant frequency (i.e., the permittivity/resistivity) of the tissue. Consequently, the scattered electromagnetic energy that the skin reflects back to the electromagnetic receiver can be analyzed relative to the incident electromagnetic energy to calculate the glucose level of blood non-invasively in real-time. In one embodiment the wearable device may transmit one or more calculated blood glucose readings wirelessly to an information handling system. The information handling system may be configured to maintain a historical record of the user's blood glucose readings and/or perform other functions, such as automatically notifying a physician via a network connection if a pre-defined blood glucose threshold level is exceeded.

The wearable device uses electromagnetic energy to measure the user's blood glucose level non-invasively, so users are much more likely to reliably comply with the system because the “pin prick” of conventional invasive blood glucose monitoring systems is not required. In addition, the wearable aspect of the device helps promote timely and accurate testing since the user can simply leave the device on their body instead of having to remember to bring the device with them. Similarly, the processor may be configured to automatically initiate blood glucose tests at regular intervals so that a busy user will not need to find time to manually initiate tests at multiple times throughout the day. A system for non-invasive blood glucose monitoring with a wearable device thus benefits the overall health and well-being of the user by ensuring accurate and timely testing and archiving of blood glucose levels.

For purposes of this disclosure, an information handling system may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, an information handling system may be a personal computer (e.g., desktop or laptop), tablet computer, mobile device (e.g., personal digital assistant (PDA) or smart phone), server (e.g., blade server or rack server), a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, touchscreen and/or a video display. The information handling system may also include one or more buses operable to transmit communications between the various hardware components.

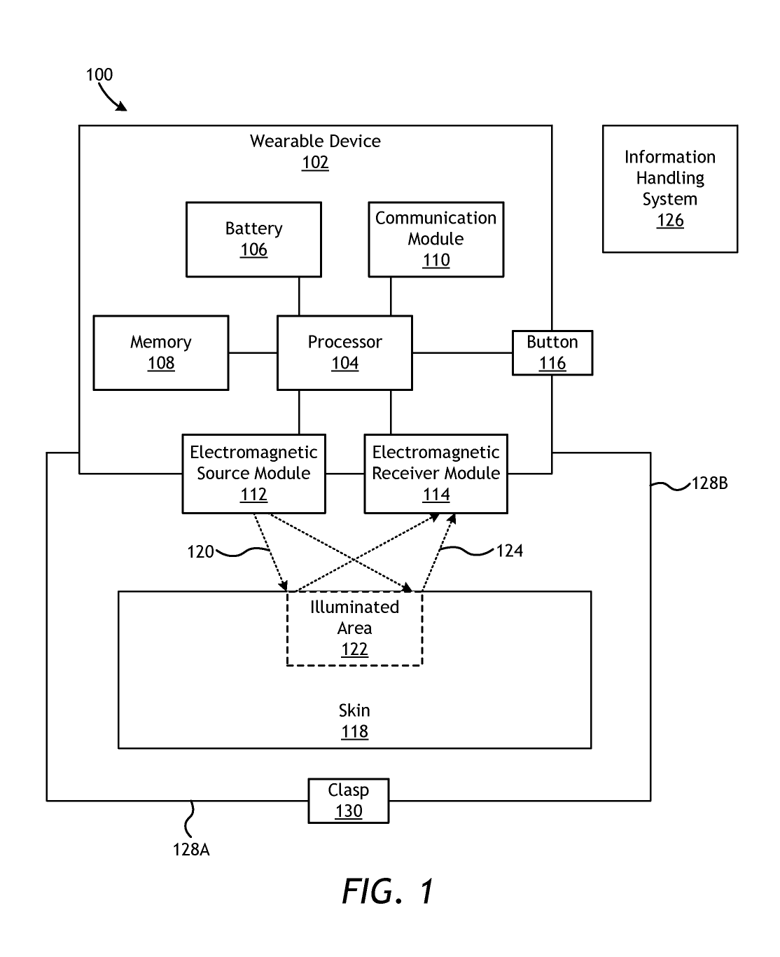

FIG. 1 is a schematic circuit diagram illustrating one embodiment of a system 100 for non-invasive blood glucose monitoring with a wearable device 102. In one embodiment wearable device 102 may include a processor 104. In an embodiment wearable device may also include one or more modules coupled to processor 104, such as a battery 106, a memory 108, a communication module 110, an electromagnetic source module 112, an electromagnetic receiver module 114, and a button 116. In various embodiments, battery 106 may be a rechargeable battery or a replaceable disposable battery. Battery 106 may be coupled to multiple components of wearable device 102 and/or processor 104 may be coupled to battery 106 such that processor 104 may distribute electrical power to the components of wearable device 102. In one embodiment processor 104 may automatically initiate one or more blood glucose tests at regular intervals. In another embodiment a user of wearable device 102 may manually initiate a blood glucose test by pressing button 116. In various embodiments, memory 108 may store calibration data, program instructions for processor 104, and/or blood glucose measurement data. The calibration data may include built-in reference values, initial self-test values, and or baseline readings of the user.

In various embodiments, electromagnetic source module 112 may be a light emitting diode (LED), a Super-Luminescent Diode (SLD), a semiconductor laser, a photo diode, a microwave emitter, a radio frequency (RF) emitter, a transducer or the like. Similarly, electromagnetic receiver module 114 may be a photodetector, a transducer, a capacitive sensor, a microwave sensor, or the like. In an embodiment, electromagnetic source module 112 and electromagnetic receiver module 114 may be oriented on wearable device 102 such that the skin 118 of a user is in proximity to electromagnetic source module 112 and electromagnetic receiver module 114. The electromagnetic source module 112 may emit incident electromagnetic energy 120 onto illuminated area 122 of the skin 118. The incident electromagnetic energy 120 may be scattered by the skin 118 of the illuminated area 122 and reflected. The reflected electromagnetic energy 124 may be measured by electromagnetic receiver module 114. Since glucose raises the refractive index of interstitial fluid and lowers the dielectric constant of blood, processor 104 may calculate a blood glucose level of the user by comparing the measured values of the received electromagnetic energy 124 to pre-defined reference values (e.g., constants) and/or the relative values of the incident electromagnetic energy 120.

In one embodiment processor 104 may use communication module 110 to transmit one or more blood glucose values to an information handling system 126. In various embodiments, information handling system 126 may be a smart phone, personal digital assistant (PDA), tablet, laptop computer, or desktop computer. In an embodiment, information handling system 126 may include an application (i.e., an “app”) configured to store a historical record of multiple blood glucose levels of the user and to generate reports that track the data levels over various time periods. In another embodiment, information handling system 126 may use an application to enable one or more qualified users (e.g., the primary user, the user's immediate family members, a nurse, or a physician) to adjust the number of intervals at which wearable device 102 is programmed to automatically initiate blood glucose tests. In yet another embodiment, an application stored in information handling system 126 may use a network connection to contact a physician or other qualified emergency contact (e.g., a family member) in response to wearable device 102 measuring a blood glucose level that is outside a pre-defined blood glucose threshold range. In an embodiment, the application may be configured for a smart phone, tablet, or computer.

In various embodiments, processor 104 and/or an application stored in information handling system 126 may selectively transmit an alert to a physician in response to one of the multiple blood glucose levels exceeding an upper threshold level, one of the multiple blood glucose levels being below a lower blood glucose threshold level, or a slope of a plotted line of historical blood glucose levels being outside a pre-defined safe blood glucose rate of change. Additionally, processor 104 and/or an application stored in information handling system 126 may be configured to automatically initiate a blood glucose test at pre-programmed time intervals, increase the frequency of the pre-programmed time intervals in response to one of the multiple blood glucose levels exceeding the upper blood glucose threshold level or being below the lower blood glucose threshold level, and/or increase the frequency of the pre-programmed time intervals in response to the slope of the plotted line of historical blood glucose levels being outside the pre-defined safe blood glucose rate of change. In an embodiment, processor 104 may initiate a blood glucose test in response to a user pressing button 116 coupled to processor 104, or in response to a user selecting a manual test initiation feature of an application of information handling system 126. In one embodiment, processor 104 may be configured to calibrate electromagnetic receiver module 114 based on a pre-defined reference value.

In an embodiment wearable device 102 may include one or more straps 128A-B connected to a clasp 130. The straps 128A-B may be configured such that the user may wear the wearable device 102 around a convenient area of the body that is desirable for non-invasive tissue testing, such as a wrist, an arm, a neck, or the like. The clasp 130 may be engaged to secure and/or remove wearable device 102 to and/or from the user, respectively. In another embodiment, wearable device 102 may be included in a patch having an adhesive material that enables wearable device 102 to be attached to the skin of a user (e.g., on the user's arm) without straps or a clasp. In one embodiment, a patch-based wearable device may use microwave electromagnetic energy to measure the resistivity/permittivity of the tissue beneath the patch and thereby calculate a blood glucose level of a user.

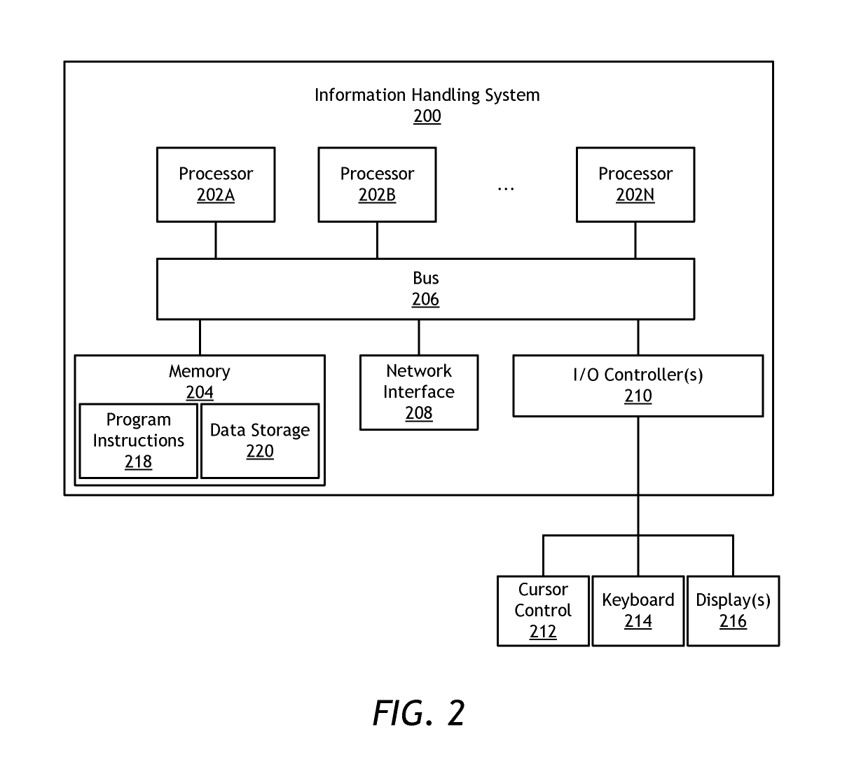

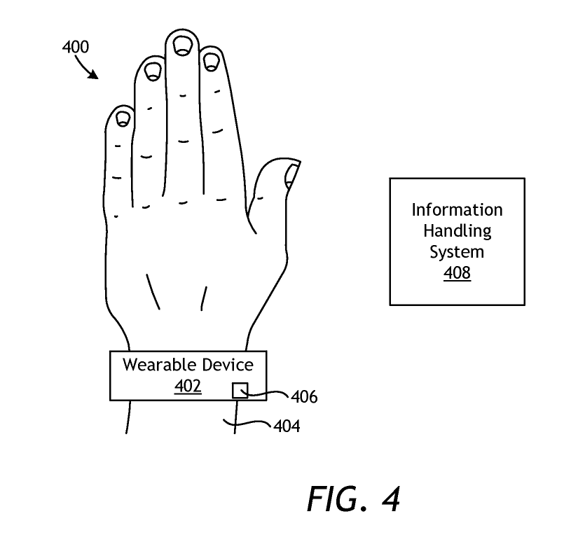

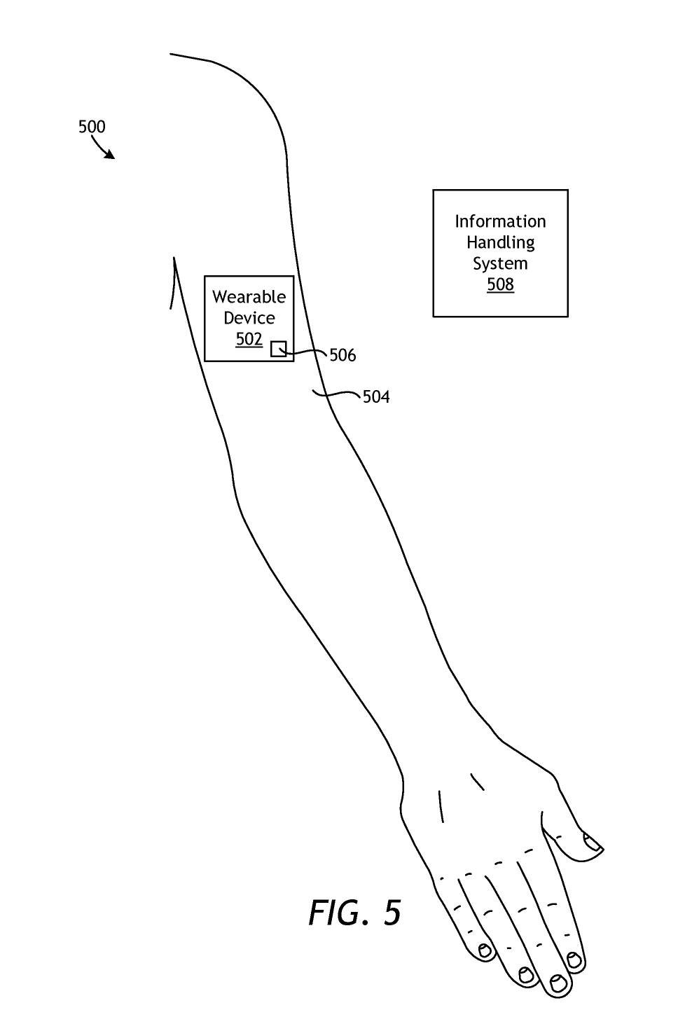

FIG. 2 is a schematic block diagram illustrating one embodiment of an Information Handling System (IHS) 200 configured for non-invasive blood glucose monitoring with a wearable device. In one embodiment, information handling system 126 of FIG. 1 may be implemented on an information handling system similar to IHS 200 described in FIG. 2. Similarly, information handling system 408 described in FIG. 4 and/or information handling system 508 described in FIG. 5 may be implemented on an information handling system similar to IHS 200 described in FIG. 2. In various embodiments, IHS 200 may be a server, a mainframe computer system, a workstation, a network computer, a desktop computer, a laptop, a tablet, a smart phone, or the like.

As illustrated, IHS 200 includes one or more processors 202A-N coupled to a system memory 204 via bus 206. IHS 200 further includes network interface 208 coupled to bus 206, and input/output (I/O) controller(s) 210, coupled to devices such as cursor control device 212, keyboard 214, and display(s) 216. In some embodiments, a given entity (e.g., information handling system 126) may be implemented using a single instance of IHS 200, while in other embodiments multiple such information handling systems, or multiple nodes making up IHS 200, may be configured to host different portions or instances of embodiments (e.g., information handling system 126).

In various embodiments, IHS 200 may be a single-processor information handling system including one processor 202A, or a multi-processor information handling system including two or more processors 202A-N (e.g., two, four, eight, or another suitable number). Processor(s) 202A-N may be any processor capable of executing program instructions. For example, in various embodiments, processor(s) 202A-N may be general-purpose or embedded processors implementing any of a variety of instruction set architectures (ISAs), such as the x86, POWERPC®, ARM®, SPARC®, or MIPS® ISAs, or any other suitable ISA. In multi-processor systems, each of processor(s) 202A-N may commonly, but not necessarily, implement the same ISA. Also, in some embodiments, at least one processor(s) 202A-N may be a graphics processing unit (GPU) or other dedicated graphics-rendering device. In an embodiment, processor 104 of FIG. 1 may be configured similarly to processor(s) 202A-N.

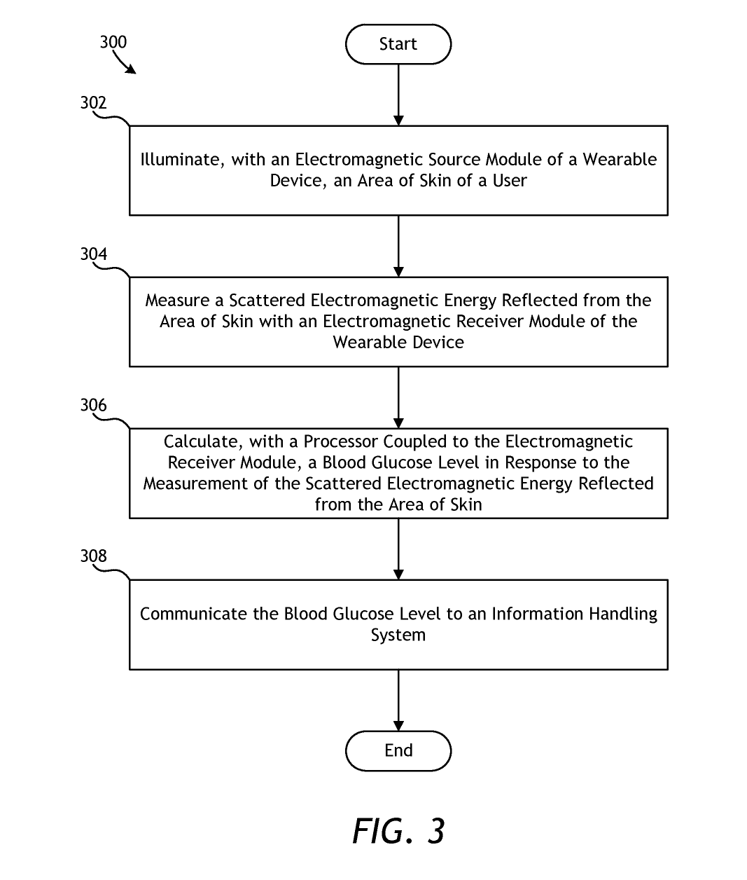

System memory 204 may be configured to store program instructions and/or data accessible by processor(s) 202A-N. For example, memory 204 may be used to store software program and/or database shown in FIG. 3. In various embodiments, system memory 204 may be implemented using any suitable memory technology, such as static random access memory (SRAM), synchronous dynamic RAM (SDRAM), nonvolatile/Flash-type memory, or any other type of memory. As illustrated, program instructions and data implementing certain operations, such as, for example, those described above, may be stored within system memory 204 as program instructions 218 and data storage 220, respectively. In other embodiments, program instructions and/or data may be received, sent or stored upon different types of IHS-accessible media or on similar media separate from system memory 204 or IHS 200. Generally speaking, a IHS-accessible medium may include any tangible, non-transitory storage media or memory media such as electronic, magnetic, or optical media-e.g., disk or CD/DVD-ROM coupled to IHS 200 via bus 206, or non-volatile memory storage (e.g., “flash” memory). In an embodiment, memory 108 of FIG. 1 may be implemented in a memory module similar to system memory 204.

The terms “tangible” and “non-transitory,” as used herein, are intended to describe an IHS-readable storage medium (or “memory”) excluding propagating electromagnetic signals, but are not intended to otherwise limit the type of physical IHS-readable storage device that is encompassed by the phrase IHS-readable medium or memory. For instance, the terms “non-transitory IHS readable medium” or “tangible memory” are intended to encompass types of storage devices that do not necessarily store information permanently, including for example, random access memory (RAM). Program instructions and data stored on a tangible IHS-accessible storage medium in non-transitory form may further be transmitted by transmission media or signals such as electrical, electromagnetic, or digital signals, which may be conveyed via a communication medium such as a network and/or a wireless link.

In an embodiment, bus 206 may be configured to coordinate I/O traffic between processor 202, system memory 204, and any peripheral devices including network interface 208 or other peripheral interfaces, connected via I/O controller(s) 210. In some embodiments, bus 206 may perform any necessary protocol, timing or other data transformations to convert data signals from one component (e.g., system memory 204) into a format suitable for use by another component (e.g., processor(s) 202A-N). In some embodiments, bus 206 may include support for devices attached through various types of peripheral buses, such as a variant of the Peripheral Component Interconnect (PCI) bus standard or the Universal Serial Bus (USB) standard, for example. In some embodiments, the operations of bus 206 may be split into two or more separate components, such as a north bridge and a south bridge, for example. In addition, in some embodiments some or all of the operations of bus 206, such as an interface to system memory 204, may be incorporated directly into processor(s) 202A-N.

Network interface 208 may be configured to allow data to be exchanged between IHS 200 and other devices, such as other information handling systems attached to information handling system 126, for example. In various embodiments, network interface 208 may support communication via wired or wireless general data networks, such as any suitable type of Ethernet network, for example; via telecommunications/telephony networks such as analog voice networks or digital fiber communications networks; via storage area networks such as Fiber Channel SANs, or via any other suitable type of network and/or protocol.

I/O controller(s) 210 may, in some embodiments, enable connection to one or more display terminals, keyboards, keypads, touch screens, scanning devices, voice or optical recognition devices, or any other devices suitable for entering or retrieving data by one or more IHS 200. Multiple input/output devices may be present in IHS 200 or may be distributed on various nodes of IHS 200. In some embodiments, similar I/O devices may be separate from IHS 200 and may interact with IHS 200 through a wired or wireless connection, such as over network interface 208.

As shown in FIG. 2, memory 204 may include program instructions 218, configured to implement certain embodiments described herein, and data storage 220, comprising various data accessible by program instructions 218. In an embodiment, program instructions 218 may include software elements of embodiments illustrated in FIG. 3. For example, program instructions 218 may be implemented in various embodiments using any desired programming language, scripting language, or combination of programming languages and/or scripting languages. Data storage 220 may include data that may be used in these embodiments such as, for example, data from processor 104 and/or electromagnetic receiver module 112. In other embodiments, other or different software elements and data may be included.

A person of ordinary skill in the art will appreciate that IHS 200 is merely illustrative and is not intended to limit the scope of the disclosure described herein. In particular, the information handling system and devices may include any combination of hardware or software that can perform the indicated operations. In addition, the operations performed by the illustrated components may, in some embodiments, be performed by fewer components or distributed across additional components. Similarly, in other embodiments, the operations of some of the illustrated components may not be performed and/or other additional operations may be available. Accordingly, systems and methods described herein may be implemented or executed with other information handling system configurations.

Embodiments of information handling system 126 described in FIG. 1, information handling system 408 described in FIG. 4, and/or information handling system 508 described in

FIG. 5 may be implemented in an information handling system that is similar to IHS 200. In one embodiment, the elements described in FIG. 1, FIG. 3, FIG. 4, and FIG. 5 may be implemented in discrete hardware modules. Alternatively, the elements may be implemented in software-defined modules which are executable by one or more of processors 202A-N, for example.

FIG. 3 is a schematic flowchart diagram illustrating one embodiment of a method 300 for non-invasive blood glucose monitoring with a wearable device. At block 302, the method 300 includes illuminating, with an electromagnetic source module of a wearable device, an area of skin of a user. As depicted in block 304, the method 300 includes measuring a scattered electromagnetic energy reflected from the area of skin with an electromagnetic receiver module of the wearable device. As shown in block 306, the method 300 includes calculating, with a processor coupled to the electromagnetic receiver module, a blood glucose level in response to the measurement of the scattered electromagnetic energy reflected from the area of skin. At block 308, the method 300 includes communicating the blood glucose level to an information handling system. In one embodiment, the information handling system may include an application (i.e., an “app”) configured to use a network connection to automatically contact a physician or other pre-defined emergency contact person (e.g., a pre-defined authorized family member) in response to the blood glucose level being beyond a pre-defined blood glucose threshold level. In an embodiment, the processor may calibrate the electromagnetic receiver module based on one or more pre-defined reference values, such as factory-programmed calibration data, baseline readings of the user, electromagnetic energy reflected from a standard reference sample, or the like.

FIG. 4 is a schematic block diagram illustrating one embodiment of an apparatus 400 for non-invasive blood glucose monitoring with a wearable device. In one embodiment the apparatus 400 may include a wearable device 402. In an embodiment the wearable device 402 may be configured to be worn around a wrist 404 of a user, such that the underside of the wearable device makes contact with the skin of the wrist. In another embodiment, wearable device 402 may be configured to be worn as a band around an arm of the user. In an embodiment the wearable device 402 may include a button 406. A user may manually initiate a blood glucose measurement by pressing button 406, thereby sending an initiation signal to a processor of wearable device 402. As depicted, the apparatus 400 also includes an information handling system 408. In an embodiment information handling system 408 may be configured similarly to IHS 200 of FIG. 2. In one embodiment information handling system 408 may be configured to receive blood glucose measurement data from wearable device 402 via a wireless connection. In another embodiment, information handling system 408 may be configured to transmit an alert or notification signal to a physician or other emergency contact (e.g., a pre-defined authorized family member) in response to wearable device 402 sending a blood glucose measurement result that is above or below a pre-defined blood glucose threshold level.

FIG. 5 is a schematic block diagram illustrating one embodiment of an apparatus 500 for non-invasive blood glucose monitoring with a wearable device. In one embodiment the apparatus 500 may include a wearable device 502. In an embodiment the wearable device 502 may be configured to be attached via an adhesive patch to an arm 504 of a user, such that the underside of the wearable device makes contact with the skin of the arm. In various embodiments, wearable device 502 may be configured to be attached to another area of skin, including but not limited to, a leg, a waist, or a rib cage of the user. In one embodiment, a patch-based wearable device 502 may use microwave electromagnetic energy to measure the resistivity/permittivity of the tissue beneath the patch and thereby calculate a blood glucose level of a user. In an embodiment the wearable device 502 may include a button 506. A user may manually initiate a blood glucose measurement by pressing button 506, thereby sending an initiation signal to a processor of wearable device 502. As depicted, the apparatus 500 also includes an information handling system 508. In an embodiment information handling system 508 may be configured similarly to IHS 200 of FIG. 2. In one embodiment information handling system 508 may be configured to receive blood glucose measurement data from wearable device 502 via a wireless connection. In another embodiment, information handling system 508 may be configured to transmit an alert or notification signal to a physician or other emergency contact (e.g., a pre-defined authorized family member) in response to wearable device 502 sending a blood glucose measurement result that is above or below a pre-defined blood glucose threshold level.

It should be understood that various operations described herein may be implemented in software executed by logic or processing circuitry, hardware, or a combination thereof. The order in which each operation of a given method is performed may be changed, and various operations may be added, reordered, combined, omitted, modified, etc. It is intended that the invention(s) described herein embrace all such modifications and changes and, accordingly, the above description should be regarded in an illustrative rather than a restrictive sense.

Although the invention(s) is/are described herein with reference to specific embodiments, various modifications and changes can be made without departing from the scope of the present invention(s), as set forth in the claims below. Accordingly, the specification and figures are to be regarded in an illustrative rather than a restrictive sense, and all such modifications are intended to be included within the scope of the present invention(s). Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements. The terms “coupled” or “operably coupled” are defined as connected, although not necessarily directly, and not necessarily mechanically. The terms “a” and “an” are defined as one or more unless stated otherwise. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements but is not limited to possessing only those one or more elements. Similarly, a method or process that “comprises,” “has,” “includes” or “contains” one or more operations possesses those one or more operations but is not limited to possessing only those one or more operations.

1. A method of non-invasive blood glucose monitoring with a wearable device, comprising:
illuminating, with an electromagnetic source module of a wearable device, an area of skin of a user;
measuring a scattered electromagnetic energy reflected from the area of skin with an electromagnetic receiver module of the wearable device;
calculating, with a processor coupled to the electromagnetic receiver module, a blood glucose level in response to the measurement of the scattered electromagnetic energy reflected from the area of skin; and
communicating the blood glucose level to an information handling system.

2. The method of claim 1, wherein the information handling system further comprises a memory configured for storing a historical record comprising a plurality of blood glucose levels.

3. The method of claim 2, wherein the information handling system further comprises a second processor configured to selectively transmit an alert to a physician in response to:
one of the plurality of blood glucose levels exceeding an upper blood glucose threshold level;
one of the plurality of blood glucose levels being below a lower blood glucose threshold level; or
a slope of a plotted line of historical blood glucose levels being outside a pre-defined safe blood glucose rate of change.

4. The method of claim 3, wherein the processor is configured to:
automatically initiate a blood glucose test at pre-programmed time intervals;
increase the frequency of the pre-programmed time intervals in response to one of the plurality of blood glucose levels exceeding the upper blood glucose threshold level or being below the lower blood glucose threshold level; and
increase the frequency of the pre-programmed time intervals in response to the slope of the plotted line of historical blood glucose levels being outside the pre-defined safe blood glucose rate of change.

5. The method of claim 1, wherein the processor initiates a blood glucose test in response to:
a user pressing a button coupled to the processor; or
a user selecting a manual test initiation feature of an application of the information handling system.

6. The method of claim 1, further comprising calibrating, with the processor, the electromagnetic receiver module based on a pre-defined reference value.

7. The method of claim 1, wherein the electromagnetic source module comprises a photodiode module or a microwave module.

8. A system for non-invasive blood glucose monitoring with a wearable device, comprising:
an information handling system; and
a wearable device comprising:
an electromagnetic source module configured to illuminate an area of skin of a user;
an electromagnetic receiver module configured to measure a scattered electromagnetic energy reflected from the area of skin;
a processor configured to calculate a blood glucose level in response to the measurement of the scattered electromagnetic energy reflected from the area of skin; and
a communication module configured to communicate the blood glucose level to the information handling system.

9. The system of claim 8, wherein the information handling system further comprises a memory configured for storing a historical record comprising a plurality of blood glucose levels.

10. The system of claim 9, wherein the information handling system further comprises a second processor configured to selectively transmit an alert to a physician in response to:
one of the plurality of blood glucose levels exceeding an upper blood glucose threshold level;
one of the plurality of blood glucose levels being below a lower blood glucose threshold level; or
a slope of a plotted line of historical blood glucose levels being outside a pre-defined safe blood glucose rate of change.

11. The system of claim 10, wherein the processor is configured to:
automatically initiate a blood glucose test at pre-programmed time intervals;
increase the frequency of the pre-programmed time intervals in response to one of the plurality of blood glucose levels exceeding the upper blood glucose threshold level or being below the lower blood glucose threshold level; and
increase the frequency of the pre-programmed time intervals in response to the slope of the plotted line of historical blood glucose levels being outside the pre-defined safe blood glucose rate of change.

12. The system of claim 8, wherein the processor initiates a blood glucose test in response to:
a user pressing a button coupled to the processor; or
a user selecting a manual test initiation feature of an application of the information handling system.

13. The system of claim 8, wherein the electromagnetic source module comprises a photodiode module or a microwave module.

14. The system of claim 8, wherein the information handling system comprises an application configured for a smart phone, tablet, or computer.

15. An apparatus for non-invasive blood glucose monitoring with a wearable device, comprising:
an electromagnetic source module configured to illuminate an area of skin of a user;
an electromagnetic receiver module configured to measure a scattered electromagnetic energy reflected from the area of skin;
a processor configured to calculate a blood glucose level in response to the measurement of the scattered electromagnetic energy reflected from the area of skin; and
a communication module configured to communicate the blood glucose level to an information handling system.

16. The apparatus of claim 15, wherein the information handling system further comprises a memory configured for storing a historical record comprising a plurality of blood glucose levels.

17. The apparatus of claim 15, wherein the electromagnetic source module comprises a photodiode module or a microwave module.

18. The apparatus of claim 15, wherein the processor is configured to:
automatically initiate a blood glucose test at pre-programmed time intervals;
increase the frequency of the pre-programmed time intervals in response to one of the plurality of blood glucose levels exceeding an upper blood glucose level or being below a lower blood glucose threshold level; and
increase the frequency of the pre-programmed time intervals in response to the slope of a plotted line of historical blood glucose levels being outside a pre-defined safe blood glucose rate of change.

19. The apparatus of claim 15, wherein the processor initiates a blood glucose test in response to:
a user pressing a button coupled to the processor; or
a user selecting a manual test initiation feature of an application of the information handling system.

20. The apparatus of claim 15, wherein the information handling system comprises an application configured for a smart phone, tablet, or computer.
